(19) United States
(12) Patent Application Publication
(10) Pub. No.: US 2023/0127176 A1
(43) Pub. Date: Apr. 27, 2023

(54) VIRUS METABOLITES DETECTION USING ENVIRONMENT AIR CAPTURE COUPLED TO A TUNABLE MEMBRANE INLET MASS SPECTROMETER

(71) Applicant: University of North Texas, Denton, TX (US)

(72) Inventor: Guido Fridolin Verbeck, IV, Lewisville, TX (US)

(21) Appl. No.: 17/907,708

(22) PCT Filed: Apr. 8, 2021
(86) PCT No.: PCT/IB2021/052930

§ 371 Date: Sep. 29, 2022

Related U.S. Application Data
(60) Provisional application No. 63/012,886, filed on Apr. 20, 2020.

(51) Int. Cl. G01N 33/497; G01N 33/00; G01N 33/68

(52) U.S. Cl. CPC G01N 33/497; G01N 33/0047; G01N 33/6848; G01N 2033/4977

(57) ABSTRACT

Embodiments of the present disclosure enable rapid detection of viruses present in ambient air flows. A fan disposed in an ambient environment may be activated to direct an air flow to an ambient inlet of an analysis device. As an ambient air flow enters an inlet of the analysis device, a heating element may introduce heat into the air flow, causing VOCs to be released. A mass spectrometer-based analysis device may be used to analyze the VOCs to detect the presence of one or more target VOCs that indicate the presence of a virus or other harmful molecule in the ambient air flow.

### CROSS-REFERENCE TO RELATED APPLICATION

The present application claims the benefit of U.S. Provisional Patent Application No. 63/012,886, filed Apr. 20, 2020 and titled “STANDOFF DETECTION OF DISEASE AND VIRUS METABOLITES USING ENVIRONMENT AIR CAPTURE COUPLED TO A TUNABLE MEMBRANE INLET MASS SPECTROMETER,” the contents of which are incorporated herein by reference in their entirety.

### TECHNICAL FIELD

The present disclosure generally relates to illness detection and more specifically to mass spectrometer techniques for mass detection of viruses and other illness causing molecules.

### BACKGROUND

While many infectious diseases and viruses are known and treatable, the recent outbreak of the COVID-19 virus pandemic has brought to light some of the shortcomings in rapid detection and screening instrumentation. These shortcomings include insufficient resources to diagnose all individuals that are at risk of infection, as well as the absence of a rapid non-invasive diagnosis method. Current diagnosis protocols involve blood withdrawal, swabs, or other highly invasive techniques and extended waiting periods may be needed to obtain the test results. The current diagnosis protocols and the extended waiting periods for obtaining results make it difficult to test large groups of individuals, which can lead to the spread of the infectious disease, wasted treatment efforts (e.g., if some form of treatment is started as a precaution before test results come back and the person is not infected), or increase the severity of the symptoms of infected persons (e.g., because the illness may become more severe while waiting on the test results to confirm the illness before beginning treatment).

### SUMMARY

The present application is directed to systems, methods, apparatuses and computer-readable storage media facilitating rapid detection and monitoring for the presence of viruses and other infectious diseases. Embodiments may utilize a fan to direct a flow of air in an ambient environment to an inlet of an analysis device. The analysis device may include a heating element downstream of the inlet to heat the ambient air flow, causing resorption of the volatile organic compounds (VOCs) present in the heated air flow. The VOCs may be provided to one or more membrane inlets of the analysis device where the VOCs may be analyzed using a mass spectrometer. The presence of one or more particular VOCs may indicate a virus or infection disease is present in the air flow.

Detection of such viruses and other harmful molecules in accordance with embodiments may be used to perform rapid testing and monitoring of large groups of persons. For example, the analysis device may be set up at the entrance to a building or other public or common area (e.g., a hallway, stairwell, elevator, parking structure, subway terminal, or other types of structures). As people enter or exit the space VOCs may be exhaled as the people breath and the fan may be configured to direct air flows containing the VOCs to the inlet for analysis. If any of the analyzed VOCs signify the presence of a virus or other type of infectious disease or harmful molecules, one or more alerts may be generated to notify one or more individuals of the presence of harmful molecules in the structure. The alerts or notifications may be used to track the spread of a virus, such as COVID-19, and identify large groups of people that may have been exposed to the virus or some other contagion, which may help mitigate the spread of the virus or contagion.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIG. 1 is a block diagram of a system for detecting health of persons using a mass spectrometer according to embodiments of the present disclosure;

FIG. 2 is block diagram of a portable mass spectrometer system according to embodiments of the present disclosure;

FIG. 3 is a diagram illustrating detection of VOCs indicative of influenza A according to embodiments of the present disclosure;

FIG. 4A is a diagram illustrating mass spectra of VOCs observed in a breath sample;

FIG. 4B is another diagram illustrating mass spectra of VOCs observed in a breath sample;

FIG. 4C is another diagram illustrating mass spectra of VOCs observed in a breath sample;

FIG. 5A is a diagram illustrating mass spectra of VOCs observed in a breath sample;

FIG. 5B is another diagram illustrating mass spectra of VOCs observed in a breath sample;

FIG. 5C is another diagram illustrating mass spectra of VOCs observed in a breath sample;

FIG. 6 is block diagram of a portable mass spectrometer system according to embodiments of the present disclosure; and

FIG. 7 is a flow diagram of a method for detecting viruses present in ambient air flows according to embodiments of the present disclosure.

### DETAILED DESCRIPTION

Various features and advantageous details are explained more fully with reference to the non-limiting embodiments that are illustrated in the accompanying drawings and detailed in the following description. Descriptions of well-known starting materials, processing techniques, components, and equipment are omitted so as not to unnecessarily obscure the invention in detail. It should be understood, however, that the detailed description and the specific examples, while indicating embodiments of the invention, are given by way of illustration only, and not by way of limitation. Various substitutions, modifications, additions, and/or rearrangements within the spirit and/or scope of the underlying inventive concept will become apparent to those skilled in the art from this disclosure.

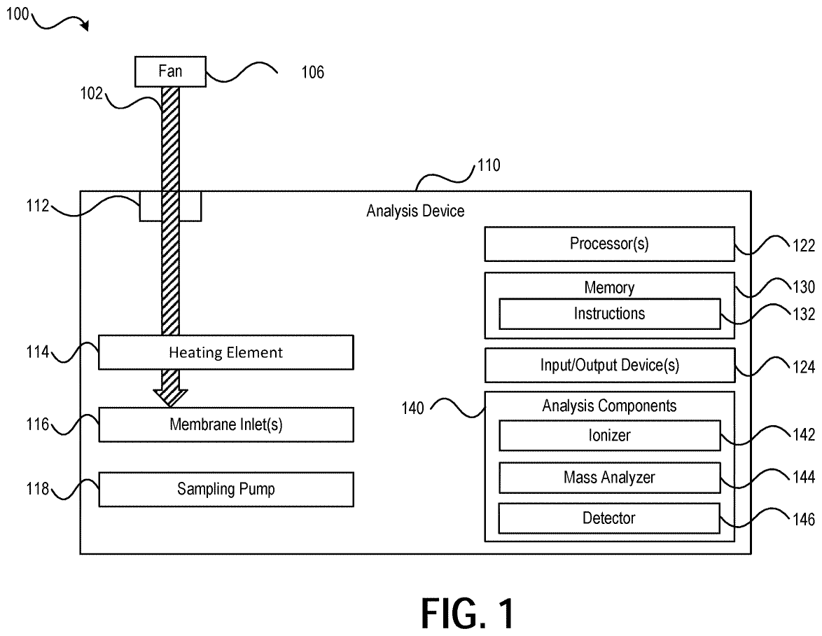

Referring to FIG. 1, a block diagram of a system for detecting health of persons using a mass spectrometer according to embodiments of the present disclosure is shown as a system 100. The system 100 may be used to detect molecules indicative of a person or persons that are suffering from an infectious disease or virus. Notably, the system 100 may provide much faster results (e.g., a few seconds) as compared to previous methods and may more easily be used to identify groups of persons that have at least been exposed to a person that is ill. For example, current portable mass spectrometric technology has the ability to present data in real-time for the detection of VOCs that may be generated by the body in the event that a healthy cell undergoes a viral infection.

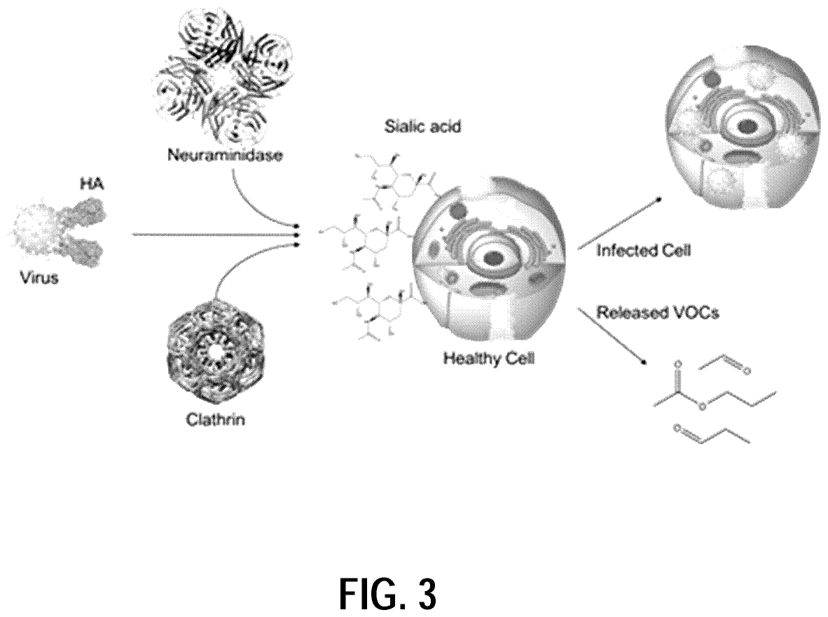

For example, in the case of Influenza A the concentration of some VOCs, such as n-propyl acetate, propanal, and acetaldehyde are found to be rather high in a specimen's breath, which may be dispersed in an ambient environment and captured using a fan that directs a flow of air from the ambient environment to an analysis device as described herein. Hemagglutinin is the surface antigen expressed by influenza A, which will bind at the cell surface level to sialic acid, as illustrated in FIG. 3. This will lead to the absorbance of the virus into the cell through a receptor induced endocytosis process, which additionally includes those actions of clathrin, enzyme neuramnidase, and many other proteins present. The formation of n-propyl acetate, whether through endocytosis, protein binding, or even cell metabolism, mimics the properties of a viral infection. Its overall formation can be said to be a result of the interactions between the cell and the virus itself, since viruses lack their own metabolic process.

The unique characteristics of certain VOCs or a combination of VOCs associated with a viral infection may enable rapid detection of viruses, such as COVID-19, through unique VOC signatures that can be used to confirm the presence of viral infections (e.g., COVID-19) in humans that may be detected using a mass spectrometer. The detection of one or more VOCs or combinations of VOCs may enable the system 100 to detect viral infections in via a non-invasive and destruction-free process. Additionally, the system 100 may enable biomarker characterization of viral infections, which would facilitate both the diagnosis and monitoring processes.

As shown in FIG. 1, the system 100 include an analysis device 110 that includes an inlet 112, a heating element 114, one or more membrane inlets 116, a sampling pump 118, one or more processors 122, a memory 130, and one or more input/output (I/O) devices 124. The one or more processors 122 may include application specific integrated circuits (ASICs), field programmable gate arrays (FPGAs), microcontrollers, central processing units (CPUs) having one or more processor cores, or other logic and circuitry configured to perform operations of the analysis device 110. The memory 130 may store instructions 132 that, when executed by the one or more processors 122, cause the one or more processors 122 to control operations of the analysis device 110 and possibly other components of the system 100, such as the fan 106, the heating element 114, or other system components utilized to analyze and identify one or more target VOCs present within the air flow 102.

The I/O devices 124 may include switches, buttons, lights, display devices, or other control elements configured to receive inputs and/or provide outputs in connection with operation of the system 100. For example, switches and/or buttons may be provided to power the system 100 on and off, identify one or more target VOCs to be identified, or other functionality and control features. Lights may be provided to indicate: the system 100 is powered on or off, the identified VOCs (e.g., different lights may be associated with different VOCs that may be identified by the system 100), or to provide other information associated with operation of the system 100. One or more display devices may additionally be provided to display information, such as to indicate the identified VOCs, indicate an operational state of the system 100 (e.g., provide information indicating one or more of the different features described above with respect to the lights or other status information), and the like.

As shown in FIG. 1, the system 100 includes a mass spectrometer device having an inlet 112 and an analysis device 110. The sampling portion 110 may include an inlet 112, a heating element 114, one or more membrane inlets 116, and a sampling pump 118. The inlet 112 may be configured to direct ambient air 102 present in an environment surrounding the system 100 (or at least ambient air in the vicinity of the inlet 112) into the inlet 112. The heating element 114 may be configured to introduce or induce heat within intake air flows. Once the flow of ambient air 102 has been received by the inlet 112, the ambient air flow 102 may be heated by the heating element 114. After being heated, the air flow 102 may be directed to the one or more membrane inlets 116.

The one or more membrane inlets 116 may be configured to allow different VOCs to pass through while excluding other VOCs from passing through. For example, membrane inlets 116 may be configured to allow VOCs of interest to be provided for analysis by analysis device 110 while VOCs that are not of interest may not pass through or be analyzed. It is noted that VOCs of interest may include VOCs that indicate the presence of a virus or other type of infectious disease. While the fan 106 may direct an air flow from the ambient environment towards the inlet 112, the sampling pump may be configured to draw the air flow 102 into the analysis device 120 under controlled conditions. For example, the sampling pump 118 may regulate the flow across the one or more membranes 116 or other chemical capture medium(s), which may prevent over-pressuring the system and under sampling of the surrounding environment. The VOCs of interest may be provided to the analysis components 140 of the analysis device 110. Exemplary operations of the analysis components 140 are described in more detail below.

The fan 106 may be configured to direct the flow of ambient air 102 towards the inlet 112. The fan 106 may be an electric fan powered by a motor. It is noted that FIG. 1 shows the fan 106 situated outside the inlet 112 for purposes of illustration, rather than by way of limitation and the fan 106 may be disposed at other locations suitable for directing the air flow 102 into the inlet 112. For example, the fan 106 may be disposed within the inlet 112 (or another location along the intake air flow pathway to the one or more membrane inlets 116) and configured to draw air into the inlet 112. It is noted that blades of the fan 106 may be oriented in different configurations depending on the particular location of the fan 106. For example, the fan blades may be oriented in a first configuration configured to push the air flow 102 towards the inlet 112 when the fan 106 is situated outside the inlet 112 and may be oriented in a second configuration configured to suck the air flow 102 into the inlet 112 when the fan 106 is situated downstream of the inlet 112. In an aspect, the different orientations of the fan blades may be achieved by a direction of rotation, such as rotating the blades in a first direction in the first orientation or a second direction in the second orientation. Alternatively, the fan blades may be rotated in the same direction in both orientations, but the fan may face opposite directions relative to the inlet in the first and second configurations. For example, air may be sucked in from one side of the fan and pushed out by the opposite side which allows the fan to push air into the inlet 112 when one side is facing the inlet (e.g., the fan is upstream of the inlet 112) and to suck air into the inlet when the other side is facing the inlet (e.g., the fan is downstream of the inlet 112).

Additionally, it is noted that FIG. 1 illustrates a single fan 106 for purposes of illustration, rather than by way of limitation and that systems according to the present disclosure may utilize more than one fan in some embodiments. For example, as described in more detail below, the fans may be placed proximate doorways to allow ambient air samples to be directed to the inlet 112 as individuals enter a building. Multiple fans may be situated proximate the doorway(s) to allow ambient air to be directed to the inlet from multiple locations, thereby increasing the likelihood that air samples containing VOCs from a person's breath are present in the air flows received by the inlet 112. To illustrate, fans may be placed at different heights to capture air flows more likely to have breath samples from persons of varying heights (e.g., a fan disposed 6 feet from the ground may not capture ambient breath samples of people under 5′6″ tall). Additionally, in an aspect, the system 100 may be configured to activate the fan(s) 106 upon a trigger event. Trigger events may include the opening of a door, a timer, actuation of an elevator door, or other types of events. In an aspect, one or more sensors may be provided to detect the triggering event. For example, a switch may be biased to an open position, but may be closed when a doorway is closed (e.g., due to pressure placed on the switch by the door when it is closed). When the doorway is opened the bias of the switch may cause it to open activate the fan to direct an ambient air flow to the inlet 112. In some aspects, the fan may be actuated after a small delay in order to allow VOCs to be dispersed into the ambient environment proximate the fan as the person enters the building. In still other aspects, the fan may always be on (at least during hours in which the building or area where the fan is disposed is open to the public).

The analysis components 140 may include an ionizer 142, a mass analyzer 144, and a detector 146. As described above, the heated air flow may be provided to the one or more membrane inlets 116, which allow the VOCs of interest to pass through the membrane inlet(s) 116. Once the VOCs pass through the membrane inlet(s) 116, they may be provided to the ionizer 142, which may be configured to ionize at least a portion of the VOCs of interest to produce one or more ionized fragments. The mass analyzer 144 may be configured to separate the one or more ionized fragments (e.g., according to a mass-to-charge ratio of the one or more ionized fragments) and the detector 146 may be configured to identify the one or more target VOCs based on the separated one or more ionized fragments. The analysis components 140 may operate under control of, or in coordination with, the one or more processors 122 of the analysis device 110. In an aspect, the analysis components 140 may include a mass spectrometer or a tetrahertz (THz) spectrometer configured to identify the one or more target VOCs present in the ambient air flow 102.

Information associated with one or more VOCs identified in the air flow 102 may be presented to a user or provided to another destination (e.g., a database stored at memory 130). For example, the information associated with the one or more VOCs may be displayed at a display device of the I/O devices 124, incorporated into a message transmitted to the user (e.g., e-mail, short messaging service (SMS), multimedia messaging service (MMS), etc.), an audio alert, a visual alert (e.g., a blinking light(s)), or other type of message or notification configured to provide information to a user about identified VOCs. In an aspect, the memory 130 may include a database that stores information regarding VOC signatures that may identify a single VOC or a combination of VOCs that, when present in the ambient air sample provided by the fan 106, indicate the presence of a virus or other harmful molecule. The VOC signatures may be generated by capturing samples of breath from persons known to be infected by a virus or other infectious disease and identifying correlations between the VOCs present in the persons' breath and the known illness. The VOC signatures may be stored in a database stored at memory 130 and used to detect the presence of the corresponding viruses by the analysis device 110. It is noted that the VOC signatures may include VOCs that may be by-product organic molecules or small molecule metabolites that occur as an artifact of cellular breakdown due to the presence of a virus or other harmful molecule(s).

The system 100 may enable rapid detection of the presence of VOCs correlated to virus infections or other contagions and harmful molecules. For example, results of an analysis cycle may be provided in real-time or near real-time, which may include providing results within a threshold time following introduction of the ambient air to the inlet by the fan. In an aspect, the threshold time may be a real-time threshold in which results are provided within 7 seconds (e.g., 1 second, 2-3 seconds, 4-5 seconds, or 6-7 seconds) of introducing the air flow into the inlet. In some aspects, the threshold time may be longer than 7 seconds (e.g., a near real-time threshold), such as 8-10 seconds, 11-20 seconds, 21-40 seconds, 41-60 seconds, or a time threshold on the order of 1-5 minutes The system 100 may enable large scale testing for the presence of a virus or other infectious disease-causing molecules in a non-invasive and cost efficient manner.

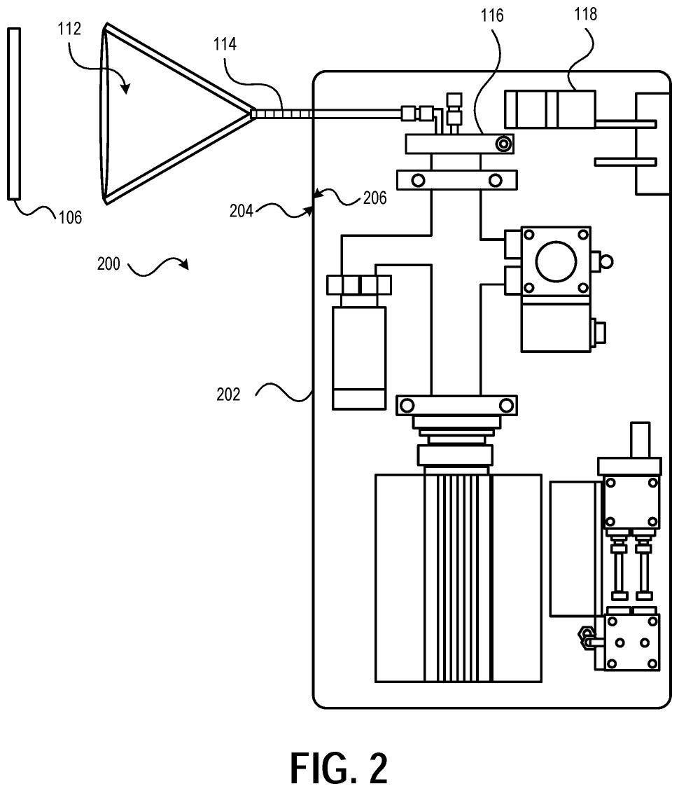

It is noted that while FIG. 1 illustrates the system 100 using a block diagram, FIG. 2 illustrates an exemplary embodiment of the system 100 as a portable mass spectrometer system according to embodiments of the present disclosure, shown as portable mass spectrometer system 200. As shown in FIG. 2, the portable mass spectrometer system 200 includes the fan 106, the inlet 112, the heating element 114, the one or more membrane inlets 116, and the sampling pump 118. The components (e.g., the inlet 112, the heating element 114, the one or more membrane inlets 116, and the sampling pump 118) of the portable mass spectrometer system 200 may be configured to be situated within a housing 202 having an exterior surface 204 and an interior surface 206. Although FIG. 2 illustrates the inlet 112 and heating element 114 as being situated outside the housing 202, embodiments are not to be limited to such arrangements and the inlet 112, the heating element 114, or both, may be disposed within or at least partially within the interior region of the housing 202.

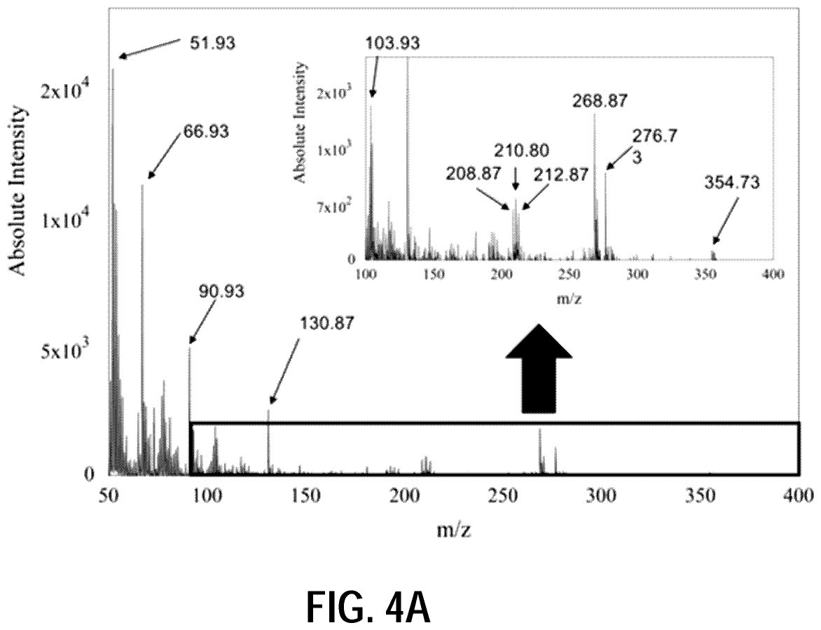

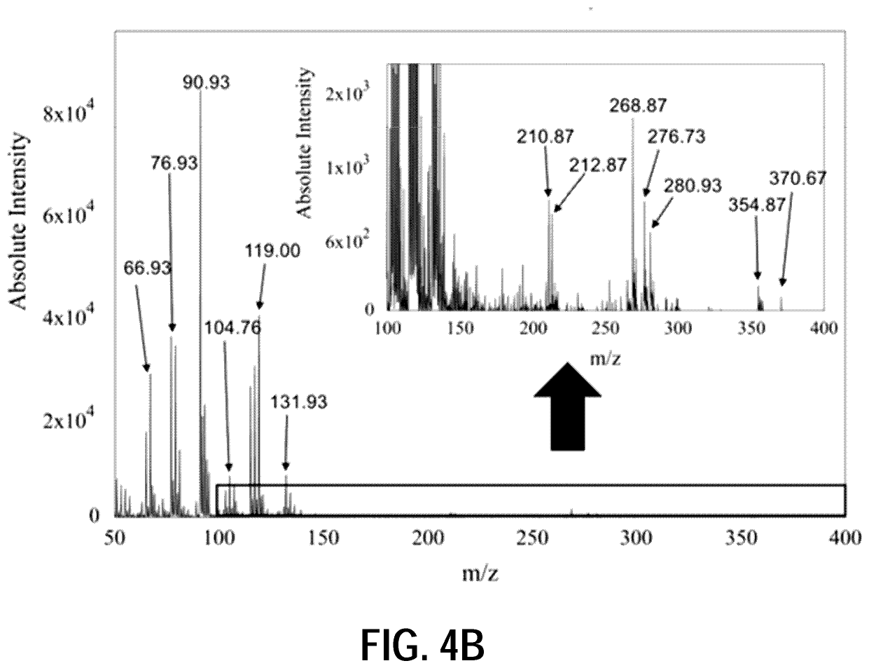

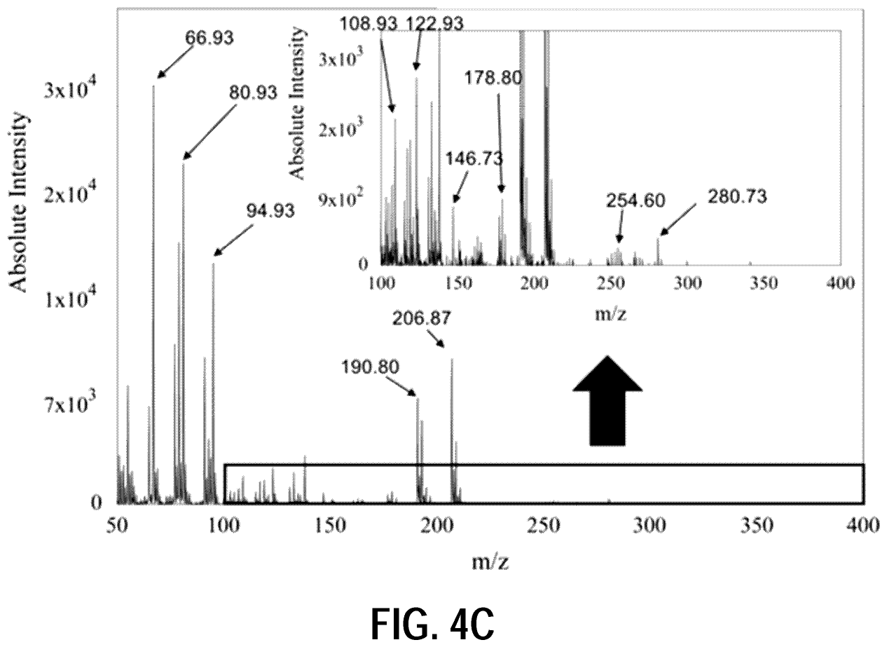

Having described in some detail the system 100 and how its various components may be used to identify VOCs of interest (e.g., VOCs indicative of a viral infection or other infection disease or illness), exemplary details of experiments demonstrating successful identification of VOCs of interest using the techniques described herein shall be described. Experiments have shown that mass spectrometers could be used to detect a person's metabolic state using the breathalyzer mass spectrometer. Healthy breath samples, breath samples from a person suffering from seasonal allergies (allergy breath), and breath samples from a person directly after washing their mouth out with Listerine were collected using a similar breathalyzer tube with a pre-concentrator attached to a PolarisQ ion trap mass spectrometer. Results of the experiments are shown in FIGS. 4A-4C. In the healthy breath sample, there is a large 51.93 m/z value, which corresponds to 1-buten-3-yne (FIG. 4A). This 1-buten-3-yne peak was not found in the allergy breath sample, where isoprene is the largest peak, followed by 1-methyl-pyrrole at 80.93 m/z and 2-ethylpyrrole at 94.93 m/z (FIG. 4B). In the mouthwash sample the isoprene was lowered as expected and other peaks were established, such as the ethylmethylsulfide peak at 76.93 m/z and 1,2,3-propanetriol at 90.93 m/z (FIG. 4C). Furthermore, larger molecular weight compounds became present, such as the octadecane peak at 254.60 m/z. The samples all show a prominent 66.93 m/z peak, which denotes isoprene. Isoprene should be found in all breath samples, and can thus, be used as a reference to ensure that the instrument is sampling ambient air containing samples of breath VOCs. Along with the potential benefits, this type of noninvasive analytical method can also contribute to both the forensic and medical fields.

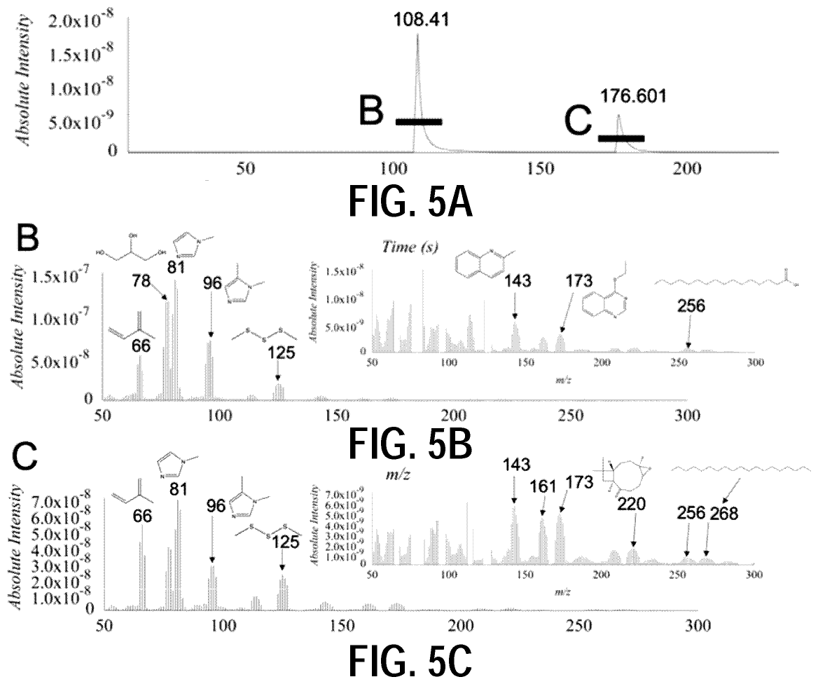

Other breath VOC metabolites have also been found in previously collected breath samples using a similar breathalyzer inlet, as shown in FIGS. 5A-5C. Metabolites such as, 1-methylimidazole, m/z 81, which is found in many caffeinated drinks, such as coffee, and dimethyl trisulfide, m/z 126, which is commonly found in the breath of people who have recently eaten cooked broccoli and onions, have been observed in breath samples of participants, as shown in FIGS. 5B and 5C. Other chemistry relating to that participant's previous meal have also been found, such as quinaldine, m/z 143, which is found in yellow food coloring associated with the cheese from the meal. Other interesting chemicals including palmitic acid, m/z 256, and nonadecane, m/z 268, have been found, which are commonly found in the saliva of humans. The most prevalent products are the result of a breakdown in lipid chemistry resulting in oxidation, ketone, and aldehyde production. Examples include acetone, acetaldehyde, and various types of benzaldehydes (e.g., tolualdehyde, and the like).

To determine viral infection of a person in the field, a rapid portable mass spectrometer, such as the system 100 of FIG. 1 or the portable mass spectrometer system 200 of FIG. 2, may be coupled to an atmospheric inlet (e.g., the inlet 112 of FIG. 1). The use of this portable system will allow for a low response time from the initial time when the ambient air flow is received. In an aspect, the response time may be 7 seconds or lower. The system may collect air flow samples using the atmospheric inlet and analyze them using a linear quadrupole mass spectrometer with the ability to measure and detect compounds that have a mass range of 1-300 with high parts per trillion (ppt) limits of detection. As described above with reference to FIG. 1, a mass spectral data library of breath chemistry (e.g., a library or database of VOC signatures) developed from preliminary data may be stored (e.g., in the memory 130 of FIG. 1) to allow for rapid system detection of viral metabolites of interest (e.g., the VOCs of interest described above with reference to FIG. 1).

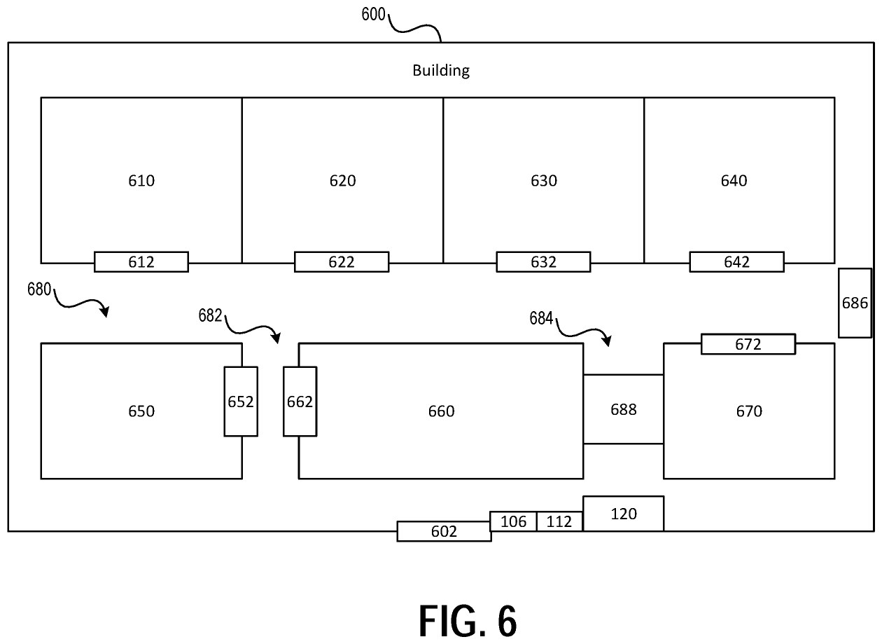

Referring to FIG. 6, a block diagram illustrating an exemplary configuration of a system for detecting VOCs of interest from ambient air flows is shown. To perform rapid detection of the presence of viruses and other illness causing molecules, a system according to the present disclosure may be disposed at entrances to building. For example, in FIG. 6, a building 600 is shown having a doorway 602, a plurality of rooms (e.g., offices, apartments, etc.) 610, 620, 630, 640, 650, 660, 670. Each of the plurality of rooms may have at least one doorway. For example, room 610 includes doorway 612, room 620 includes doorway 622, room 630 includes doorway 632, room 640 includes doorway 642, room 650 includes doorway 652, room 660 includes doorway 662, room 670 includes doorway 672. Additionally, building 600 may include one or more hallways 680, 682, 684, one or more stairways 686, and one or more elevators 688.

A mass spectrometer-based system according to the present disclosure, such as the system 100 of FIG. 1 or the system 200 of FIG. 2, may be disposed proximate the doorway 602. As the doorway 602 opens and persons enter the building 602, the fan 106 may provide an air flow to the inlet 112 for analysis by the analysis device 120, as described above with reference to FIG. 1. As persons enter the doorway 602 their breath may release VOCs into the ambient air within the building 600 and the air flow provided to the inlet 112 may contain VOCs indicative of a person suffering a virus, such as persons suffering from COVID-19, which may be detected by the analysis device 120. When viruses are detected to be present in the input air flow, an alert may be generated, such as an e-mail notification, an SMS message, a text message, an MMS message, a page, an automated voice response system message, and the like. The alert may be configured to notify persons of interest that a person suffering from a virus or other infectious disease has entered the building 600. This may enable more efficient identification and tracking of persons that may have come into contact with or been exposed to of viruses and other harmful molecules, which can lead to more rapid containment and mitigate spreading of the viruses and other harmful molecules.

To illustrate, suppose that between the hours of 9:00 AM and 11:00 AM the analysis device 120 does not detect the presence of any VOCs of interest as people enter and exit the building 600, but at 11:05 AM a VOC indicating a virus is detected. As described above, an alert may be generated and personnel may be notified that a person has entered building 600 while suffering from a viral infection. Depending on the particular virus identified, the notified personnel may then take action to prevent further individuals from entering the building (e.g., to prevent additional persons from coming into contact with the detected virus) and to identify persons that are in the building (e.g., to facilitate monitoring of those individuals for signs that they have become infected by the detected virus).

As the COVID-19 pandemic has proven, one of the difficulties in containing a virus can be the containment and mitigation of the spread of the virus. This is because many people may be asymptomatic, causing them to believe they are not suffering from the virus, but those people may be spreading the virus to others who develop more serious and even life threatening symptoms. Despite not showing physical symptoms, such as a cough, a fever, etc., asymptomatic persons may still have VOCs indicative of the virus in their breath, which may be detected by the systems and methods disclosed herein. Although not shown in FIG. 6, buildings may contain multiple entrances/doorways and analysis devices and systems of the present disclosure may be disposed at each entrance to provide more efficient analysis of the persons entering and exiting the building 600. Furthermore, analysis devices and systems may be disposed at the entrances to internal portions of the building, such as offices, hallways of apartment buildings, public bathrooms, elevators, stairways, etc. to enable identification of specific areas where infected persons were present and areas where no viruses were detected. Moreover, once a virus is detected, the notified personnel may be able to identify the location where the an infected person has been present, thereby allowing more efficient cleaning of those spaces (e.g., the entire building may not have been exposed to the virus and so only portions of the building may need to be disinfected).

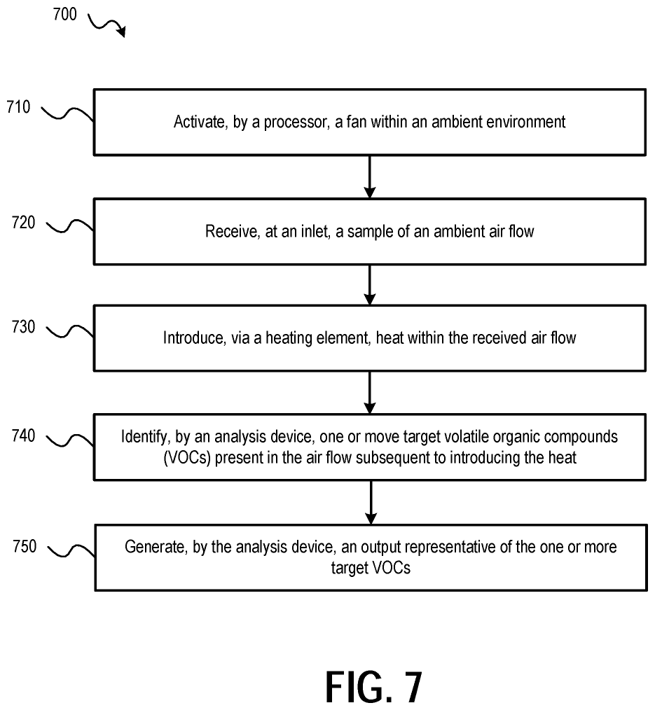

Referring to FIG. 7, a flow diagram of a method for detecting viruses present in ambient air flows according to embodiments of the present disclosure is shown. In an aspect, the method 700 may be performed by an analysis device, such as the analysis device 100 of FIG. 1 or the analysis device 200 of FIG. 2. Additionally, the method 700 may be used to detect viruses present in a building, as described with reference to FIG. 6. In an aspect, steps of the method 700 may be stored as instructions (e.g., the instructions 132 of FIG. 1) that, when executed by one or more processors (e.g., the one or more processors 122 of FIG. 1), cause the one or more processors to perform the steps of the method 700 for detecting viruses present in an ambient environment.

At step 710, the method 700 includes activating, by one or more processors, a fan disposed within an ambient environment. In an aspect, the fan may be the fan 106 of FIGS. 1, 2, and 6. At step 720, the method 700 includes receiving, at an inlet, a sample of an ambient air flow. As described above, the fan may be configured to direct a flow of ambient air within an environment to an inlet of an analysis device. At step 730, the method 700 includes introducing, via a heating element, heat within the received air flow. At step 740, the method 700 includes identifying, by an analysis device, one or more target volatile organic compounds (VOCs) present in the air flow subsequent to introducing the heat. In an aspect, the one or more target VOCs may correspond to VOCs indicative of a virus of a plurality of viruses. At step 750, the method 700 includes generating, by the analysis device, an output representative of the one or more target VOCs. As described above, the output representative of the one or more target VOCs comprises information that indicates the identified virus of the plurality of viruses and may be transmitted via a variety of communication mediums (e.g., text/SMS/MMS messages, e-mail, automated voice response (AVR) messages, a pager, and the like) to one or more users. The output may notify the user(s) that a virus has been detected as present, which may allow the user to initiate a response, such as notifying individuals present in the building, recording the names of the people present in the building proximate the time when the virus was detected, or other operations that may facilitate tracking and monitoring of the virus. In an aspect, the output may identify one or more locations where the virus was detected, such as to identify a doorway proximate the analysis device that detected the virus. In an aspect, information associated with identification of the virus may be stored at a database, as described above with reference to FIG. 1.

The ability to obtain such a wealth of chemical information from the exhaled breath of a viral infected individual also means that there are inevitable legal ramifications of the technology: right to privacy vis-à-vis confidentiality of medical health records. While exhaled breath samples are used for identification purposes by the health system, chemical biomarkers can contain personal information about each individual within the chemistry present, including gender and drug metabolites. Special protocols may be utilized to ensure that any medical information obtained from exhaled breath present in ambient air samples complies with all state and federal privacy laws related to health care.

The disclosed identification methodology could vastly aid in the identification of medical health issues, including bacterial and metabolic states. The current invasive techniques can be improved by gaining valuable chemical information using this non-invasive method. The data collected can also be collected and compared for future usage.

Although embodiments of the present application and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps. Moreover, the scope of the present application is not intended to be limited to the particular embodiments of the process, machine, manufacture, composition of matter, means, methods and steps described in the specification.

1. A method for detecting viruses present in ambient air flows, the method comprising:
activating, by one or more processors, a fan disposed within an ambient environment;
receiving, at an inlet, a sample of an ambient air flow, wherein the ambient air flow is directed to the inlet by the fan;
introducing, via a heating element, heat within the received air flow;
identifying, by an analysis device, one or more target volatile organic compounds (VOCs) present in the air flow subsequent to introducing the heat, wherein the one or more target VOCs correspond to VOCs indicative of a virus of a plurality of viruses; and
generating, by the analysis device, an output representative of the one or more target VOCs, wherein the output representative of the one or more target VOCs comprises information that indicates the identified virus of the plurality of viruses.

2. The method of claim 1, further comprising determining a location associated with the identified virus.

3. The method of claim 1, further comprising transmitting the output to one or more users.

4. The method of claim 1, further comprising storing information associated with identification of the virus at a database.

5. A system for detecting viruses present in ambient air flows, the system comprising:
an inlet;
a fan configured to direct an ambient air flow to the inlet;
a heating element configured to heat the air flow downstream of the inlet;
an analysis device configured to:
identify one or more target volatile organic compounds (VOCs) present in the air flow subsequent to introducing the heat, wherein the one or more target VOCs correspond to VOCs indicative of a virus of a plurality of viruses; and
generate, by the analysis device, an output representative of the one or more target VOCs, wherein the output representative of the one or more target VOCs comprises information that indicates the identified virus of the plurality of viruses.

6. The system of claim 5, wherein the analysis device comprises:
an ionizer;
a mass analyzer; and
a detector.

7. The system of claim 6, wherein the ionizer is configured to ionize at least a portion of the VOCs of interest to produce one or more ionized fragments.

8. The system of claim 7, wherein the mass analyzer is configured to separate the one or more ionized fragments.

9. The system of claim 8, wherein the detector is configured to identify the one or more target VOCs based on the separated one or more ionized fragments.

10. The system of claim 5, wherein the analysis device comprises a sampling pump.

11. The system of claim 5, wherein the fan is situated proximate an entry to a building and is activated to provide, to the inlet, air flows containing VOCs associated with one or more persons entering or exiting the entry of the building.

12. The system of claim 5, further comprising one or more processors configured to control operations of the analysis device.

13. The system of claim 12, wherein the one or more processors are configured to determine a location associated with the identified virus.

14. The system of claim 12, wherein the one or more processors are configured to transmit the output to one or more users.

15. The system of claim 12, wherein the one or more processors are configured to store information associated with identification of the virus at a database, and wherein the database is stored at a memory communicatively coupled to the one or more processors.

16. The system of claim 5, wherein the plurality of viruses includes COVID-19.

17. A non-transitory computer-readable storage medium storing instructions that, when executed by one or more processors, cause the one or more processors to perform operations for detecting viruses present in ambient air flows, the operations comprising:
activating a fan disposed within an ambient environment;
receiving a sample of an ambient air flow at an inlet, wherein the ambient air flow is directed to the inlet by the fan;
activating a heating element to heat the received air flow;
identifying one or more target volatile organic compounds (VOCs) present in the air flow subsequent to introducing the heat, wherein the one or more target VOCs correspond to VOCs indicative of a virus of a plurality of viruses; and
generating an output representative of the one or more target VOCs, wherein the output representative of the one or more target VOCs comprises information that indicates the identified virus of the plurality of viruses.

18. The non-transitory computer-readable storage medium of claim 17, the operations further comprising determining a location associated with the identified virus.

19. The non-transitory computer-readable storage medium of claim 17, the operations further comprising at least one of transmitting the output to one or more users and storing information associated with identification of the virus at a database.

20. The non-transitory computer-readable storage medium of claim 17, wherein the plurality of viruses include COVID-19.
